(19) United States
(12) Patent Application Publication
(10) Pub. No.: US 2003/0204217 A1
(43) Pub. Date: Oct. 30, 2003

(54) MRI-safe cardiac stimulation device

(76) Inventor: Wilson Greatbatch, Akon, NY (US)

Correspondence Address: GREENWALD & BASCH, LLP, 349 WEST COMMERCIAL STREET, SUITE 2490, EAST ROCHESTER, NY, 14445, US

(21) Appl. No.: 10/132,851

(22) Filed: Apr. 25, 2002

(51) Int. Cl. A61N 1/372

(52) U.S. Cl. 607/36

(57) ABSTRACT

An MRI-safe cardiac stimulation device includes a voltage discharge unit adapted to generate voltage pulses, a pair of implantable electrodes connected to deliver voltage pulses from the voltage discharge unit to implanted cardiac tissue, and an electrode isolation system S adapted to electrically isolate the electrodes from the voltage discharge unit during time intervals between the voltage pulses, the electrode isolation system being responsive to the voltage pulses to connect the voltage discharge unit to the electrodes during the voltage pulses.

### BACKGROUND OF THE INVENTION

1. Field of the Invention

The present invention relates to cardio-stimulation equipment designed for compatibility with MRI diagnostic apparatus. More particularly, the invention concerns an MRI-safe defibrillator.

2. Description of Prior Art

By way of background, MRI compatible cardio-stimulators, namely pacemakers, have been disclosed for both implantable and wearable in commonly assigned, copending application Ser. Nos. 09/864,944 and 09,865,049, both filed on May 24, 2001, and copending Ser. Nos. 09/885,867 and 09/885,868, both filed on Jun. 20, 2001. In the aforementioned copending patent applications, whose contents are fully incorporated herein by this reference, the disclosed pacemakers feature photonic catheters carrying optical signals in lieu of metallic leads carrying electrical signals in order to avoid the dangers associated with MRI-generated electromagnetic fields. Electro-optical and opto-electrical transducers are used to convert between electrical and optical signals. In particular, a laser diode located in a main pacemaker enclosure at a proximal end of the photonic catheter is used to convert electrical pulse signals generated by a pulse generator into optical pulses. The optical pulses are carried over an optical conductor situated in the photonic catheter to a secondary housing at the distal end of the photonic catheter, where they are converted by a photo diode array into electrical pulses for cardiac stimulation.

Despite the advances in pacemaker MRI compatibility offered by the cardio-stimulation devices of the above-referenced copending applications, there remains a problem of how to provide high voltage cardio-stimulation for defibrillation or other purposes. In particular, the photonic solution is not practical for defibrillators because the power level of the defibrillator pulse (typically about 4 kilowatts) is too high to handle with semiconductor elements. Metallic lead wires are thus required. However, the use of such materials presents its own complications, as explained in the above-cited references. The problem is three-fold. First, metallic lead wires of the type conventionally used to connect a defibrillator to an implanted heart can act as an antenna, picking up voltages and currents induced from the intense electromagnetic fields of the MRI machine. Secondly, the induced currents from the intense electromagnetic fields can be strong enough to heat the terminal ends of the defibrillator leads sufficiently to actually scar the heart. Also, the induced voltages can be conducted directly into the defibrillator and may disrupt, damage, or even destroy the sensitive semiconductor circuitry there. Lastly, the metal of the leads can produce a shadow which can be strong enough to adversely affect the diagnostic accuracy of the MRI image, particularly if the metallic material comprising the catheter is ferromagnetic (made of iron, nickel, cobalt, or alloys of any of them). Thus, to be MRI compatible, any implanted portion of a defibrillator system must contain no ferromagnetic materials, must contain only a minimal mass of any metal of any kind and must have no circuits containing long electrical pathways that can act as antennae. The foregoing poses a non-trivial design problem in the cardiac stimulation equipment art.

### SUMMARY OF THE INVENTION

The foregoing problem is solved and an advance in the art is provided by a novel MRI-safe cardiac stimulation device. The device includes a voltage discharge unit adapted to provide voltage pulses for defibrillation or other purposes. Two implantable electrodes are connected to deliver voltage pulses from the voltage discharge unit to implanted cardiac tissue. An electrode isolation system is adapted to electrically isolate the electrodes from the voltage discharge unit during time intervals between the voltage pulses. The electrode isolation system is responsive to the voltage pulses to connect the voltage discharge unit to the electrodes during the voltage pulses and to disconnect the voltage discharge unit from the electrodes between pulses. In this way, the implantable portion of the device that is susceptible to MRI-induced fields will be prevented from causing damage to tissue and circuitry alike.

In preferred embodiments of the invention, the electrode isolation system is implemented using one or more voltage-activated switches that are adapted to close in response to an applied voltage differential. The required voltage differential is preferably in excess of a voltage that could be induced into the device by an MRI apparatus but less than or equal to the level of operational voltages.

Various species of voltage-activated switches may be used for the electrode isolation system, including spark gap devices such as gas discharge tubes. The one or more switches may include a first voltage-activated switch disposed between a first side of the voltage discharge unit and a first one of the electrodes. Alternatively, the one or more switches may include a second voltage-activated switch disposed between a second side of the voltage discharge unit and a second one of the electrodes. In still another configuration, the one or more voltage-activated switches may include both of the above-described first and second voltage-activated switches.

The voltage discharge unit may include a capacitor adapted for connection to a charging source and a switch adapted to switch between a first switching state in which the charging source is connected to charge the capacitor and a second switching state in which the capacitor is connected to deliver voltage pulses to the electrodes. The charging source may comprise either a portable or fixed device and the switch may be adapted for either manual or automated control.

The electrodes are preferably mounted at the distal end of an implantable catheter made of a body-compatible material. The voltage discharge unit and the electrode isolation system can be installed in a housing that is located at the proximal end of the photonic catheter. The housing could be adapted to remain internally within a body in which the photonic catheter and electrodes are indwelling, or it could be external to the body, and possibly wearable. The electrodes are connected to the voltage discharge unit via electrical leads disposed in the catheter. Preferably, the leads will be made from a material of low magnetic susceptance and sized so as to minimize MRI image disruption.

In still other embodiments, the cardiac stimulation device of the invention can be combined with a photonic pacemaker and/or a photonic cardioverter having a wearable or implantable housing and a photonic catheter. The voltage discharge unit and the electrode isolation system of the invention could be placed in the wearable or photonic housing, and the electrodes could be disposed at the distal end of the photonic catheter. Fiber optic elements in the photonic catheter would deliver optical signals that are converted to electrical impulses to drive the electrodes for pacing or cardioverter functions. Electrical lead elements in the photonic catheter would deliver electrical signals that drive the electrodes at higher voltages for defibrillation or other cardio-stimulation purposes. Additional fiber optic elements can be provided in the photonic catheter to deliver optical sensing signals (such as R-wave amplified signals) from the distal end of the photonic catheter to the wearable or implantable housing. The sensing signals could be used to control the switch that connects the voltage discharge unit to the electrodes.

### BRIEF DESCRIPTION OF THE DRAWINGS

The foregoing and other features and advantages of the invention will be apparent from the following more particular description of preferred embodiments of the invention, as illustrated in the accompanying Drawings in which:

FIG. 1 is a schematic view of a cardiac stimulation device constructed in accordance with the present invention using one voltage-activated switch;

FIG. 2 is a schematic view of a defibrillator constructed in accordance with the present invention using two voltage-activated switches;

FIG. 3 is a diagrammatic view of a external, manually controlled implementation of the cardiac-stimulation device of FIG. 1; and

FIG. 4 is a diagrammatic view of an implantable, automatically controlled implementation of the cardiac stimulation device of FIG. 1.

### DETAILED DESCRIPTION OF PREFERRED EMBODIMENTS

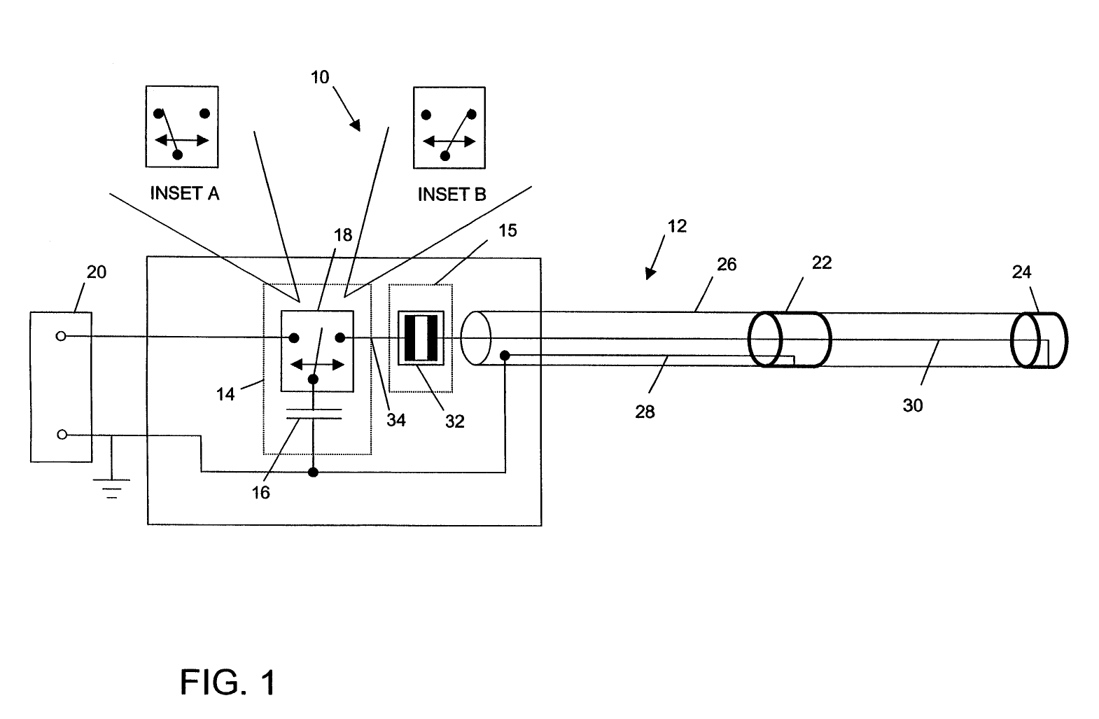

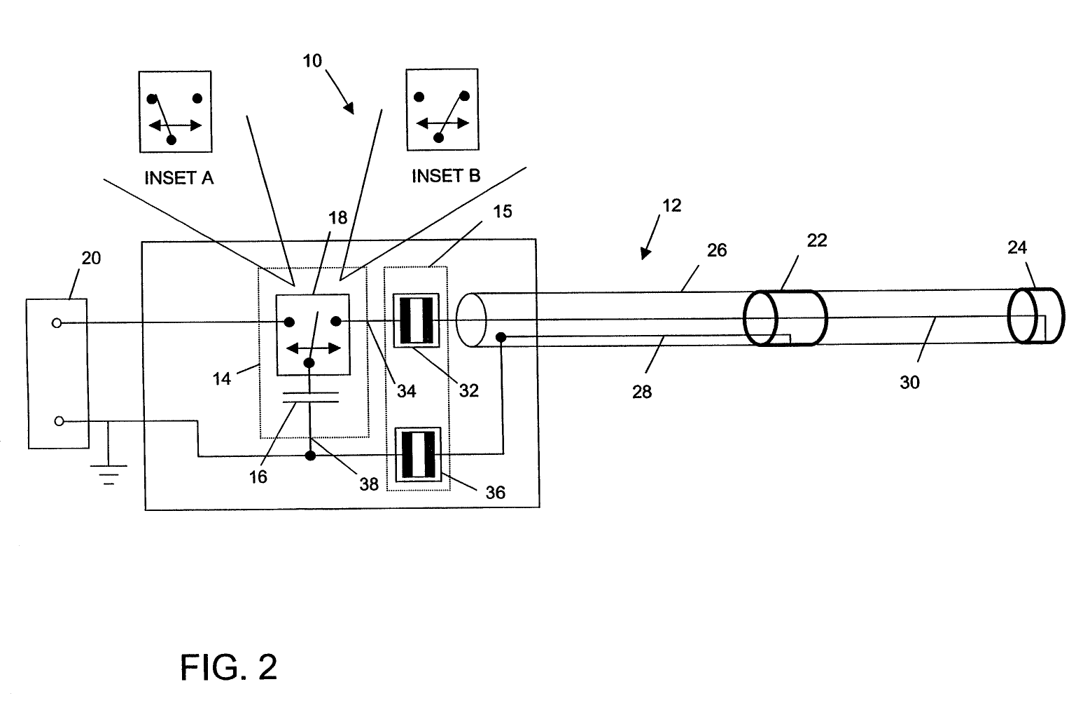

Turning now to the Drawings wherein like reference numerals signify like elements in all of the several views, FIGS. 1 and 2 show a cardiac stimulation device 10 that is designed in accordance with the invention. Summarizing in advance, the device 10 principally includes an indwelling cardiac catheter 12, a voltage discharge unit 14 adapted to provide periodic voltage pulses to the catheter 12, and an electrode isolation system 15 disposed between the voltage discharge unit and the catheter's proximal end.

The voltage discharge unit 14 can be implemented in a variety of ways. FIGS. 1 and 2 show one possible embodiment in which the voltage discharge unit 14 is provided by a capacitor 16 and a switch 18. The switch 18 is shown to be of the single pole, double throw variety. It could be a manual switch in embodiments of the device 10 where the voltage discharge unit 14 is designed to operate externally of a patient's body, or an automatically-controlled switch for embodiments of the device 10 in which the voltage discharge unit 14 is designed for implantable use (see below).

In a first switch state, shown by inset A of FIGS. 1 and 2, the switch 18 connects the capacitor 16 to a charging source 20. The charging source 20 can be implemented using either a portable power device or a fixed power device depending on design preferences and whether the voltage discharge unit 14 is intended for external or implantable use. An exemplary portable power source could comprise one or more low voltage batteries and a d.c.-d.c. converter to develop the required voltage. An exemplary fixed power source could comprise an a.c-d.c. converter powered by an a.c. line source. The voltage output of the charging source 20 will depend on the desired biological effect. For defibrillation, a voltage level of about 800 volts is preferred.

Periodically, when it is desired to deliver a voltage pulse, the switch 18 will be switched to a second switch state, as shown by inset B of FIGS. 1 and 2. This will cause the capacitor 16 to rapidly discharge through the electrode isolation system 15 into the proximal end of the catheter 12 (as described in more detail below). The catheter 12 includes a pair of implantable electrodes 22 and 24 that are situated at the distal end of an implantable catheter body 26. The electrode 22 represents a ring electrode and the electrode 24 represents a tip electrode. Both are preferably made from a material of low magnetic susceptance, such as titanium, platinum, or alloys thereof. The catheter body 26 can be made of silicone rubber, polyurethane, polyethylene or other suitable biocompatible polymer having the required mechanical and physiological properties.

The electrodes 22 and 24 are respectively connected via electrical leads 28 and 30 to deliver voltage pulses from the voltage discharge unit 14 to implanted cardiac tissue. Like the electrodes 22/24, the electrical leads 28/30 are preferably made from a material having low magnetic susceptance, such as titanium, platinum, or alloys thereof. The electrical leads 28/30 are also preferably sized so as to minimize sized MRI image disruption. This can be done by making them as thin as possible.

Notwithstanding the foregoing precautions, it will be appreciated that the electrodes 22/24 and the electrical leads 28/30 could couple RF energy from an MRI imaging apparatus into the cardiac stimulation device 10, with possible consequent adverse effect on device components (such as the switch 18) and/or insult to a patient's implanted cardiac tissue. In order to minimize the likelihood of such adverse consequences, the cardiac stimulation device 10 is provided with the electrode isolation system 15. The electrode isolation system 15 is designed to electrically isolate the electrodes 22/24 from the voltage discharge unit 14 during time intervals between the voltage pulses that are output by the voltage discharge unit. The electrode isolation system 15 responds to the voltage pulses by temporarily establishing a circuit connection between the voltage discharge unit 14 and the electrodes 22/24 during the time interval that the voltage pulses are active.

The electrode isolation system 15 can be implemented in a variety of ways. FIGS. 1 and 2 illustrate two exemplary configurations in which one or more voltage-activated switches are used. In particular, FIG. 1 shows an implementation of the electrode isolation system 15 in which the one or more voltage-activated switches comprise a first voltage-activated switch 32 disposed between a first side 34 of the voltage discharge unit 14 and a first one of the electrodes 22/24, namely, the tip electrode 24. FIG. 2 shows an alternative implementation of the electrode isolation system 15 in which the one or more voltage-activated switches include the first voltage-activated switch 32 of FIG. 1, and a second voltage-activated switch 36 disposed between a second side 38 of the voltage discharge unit 14 and a second one of the electrodes 22/24, namely, the ring electrode 24. Although not shown, another implementation of the electrode isolation system 15 could utilize the voltage-activated switch 36 by itself, without using the voltage-activated switch 32.

The voltage-activated switches 32 and 36 are preferably designed so that the voltage differential required to cause them to close is in excess of a voltage that would be induced into the device 10 by an MRI apparatus, but less than or equal to the level of the voltage pulses delivered by the voltage discharge unit 14. So long as this requirement is met, there are various species of voltage-activated switches that may be used, including spark gap devices such as gas discharge tubes, and semiconductor devices such as zener diodes (preferably arranged back-to-back for a.c. signal blockage) and metal oxide varisters (MOVs). Due to the relatively low voltage drop characteristics of the spark gap devices in comparison to the higher voltage drop characteristics of the semiconductor devices, spark gap devices are the preferred choice for implementing the voltage-activated switches 32 and 36.

Spark gap isolation switches are conventionally known for use as protective over-voltage “snubbers.” They are designed to arc at a design voltage that is normally higher than the circuit components being protected. As such, spark gap devices are typically connected to bypass one or more circuit elements rather than being integrally incorporated in a circuit such as the device 2.

One commercially available source of spark gap devices that may be used to provide the voltage-activated switches 32 and 36 of the electrode isolation system 15 is Citel, Inc., of 1111 Parkcentre Blvd., Suite 340, of Miami, Fla. 33169. This company offers a variety of spark gap products that are referred to as “surge arrester gas tubes.” Citel's “BH” line of surge arrester gas tube part numbers comprises a set of ceramic gas discharge tubes having nominal breakdown voltages ranging from 350-2500 volts. Each such device has a ceramic body charged with a proprietary gas, and an electrical contact plates on ends thereof In an experimental implementation of the invention where the device 10 was designed or use as a defibrillator adapted to deliver approximately 800 volt discharge pulses, two 230 volt “BA” model ceramic gas discharge tubes were used to implement the voltage-activated switches 32 and 36. Testing has shown that these gas discharge tubes are capable of repeated cycling at the required 800 volt level without significant break down. The tested ceramic gas discharge tubes have been found to arc at about 200 volts and to produce a low-resistance plasma for as long as their spark gaps remain conductive. During the time that the gas discharge tubes are arcing, the capacitor 16 discharges into the catheter 12. During each pulse, as the capacitor's voltage output drops off to the threshold of the gas discharge tubes, which has been measured at approximately 70 volts, their spark gaps cease conducting and revert to a series resistance of many megaohms. This produces an open-circuit condition at the proximal end of the catheter 12 that should prevent the catheter's electrical leads 28 and 30 from acting as antennae in the presence of intense electromagnetic fields such as those generated by an MRI imaging system.

Pulses of 800 volts (at about 40 joules) and having a pulse width of about 15-20 milliseconds were produced when the capacitor 16 of the above-described experimental defibrillator had a capacitance rating of 124 microfarads and the catheter 12 was connected to a 40 ohm load to simulate implanted conditions. The 15-20 millisecond pulse length represents the discharge time required for the capacitor 16 to discharge from its 800 volt fully charged state to the 70 volt cut-off voltage of the gas discharge tubes used to implement the switches 32 and 36. This is deemed acceptable for defibrillation purposes.

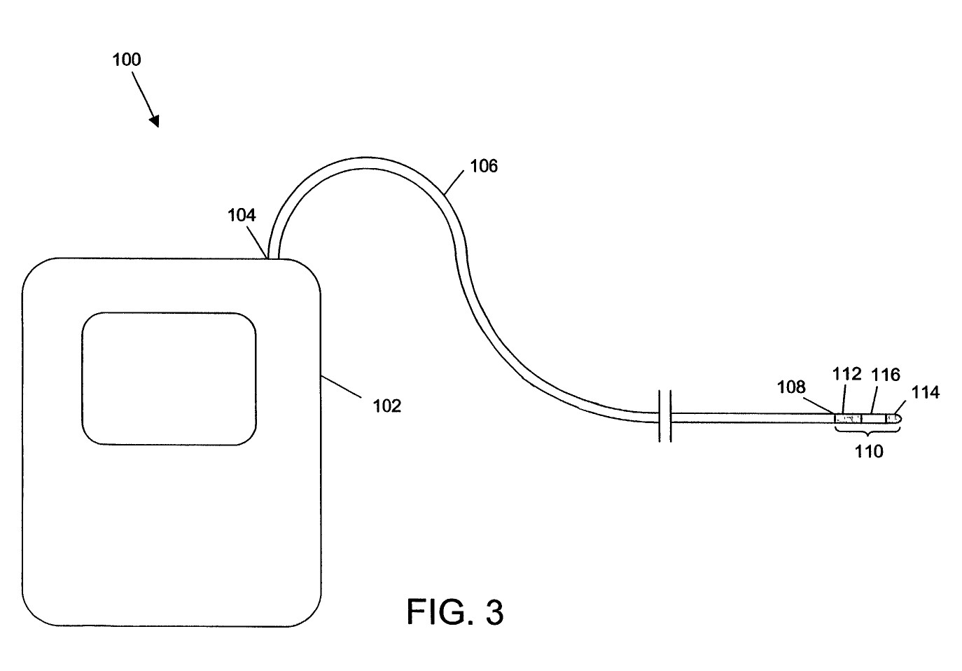

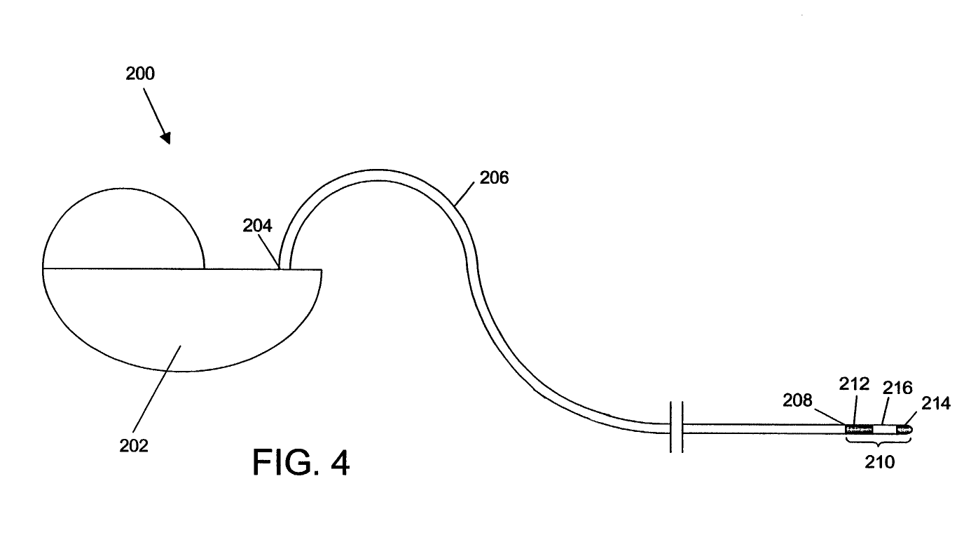

Turning now to FIGS. 3 and 4, two exemplary embodiments of the invention are shown in which the circuit components of the device 10 are respectively incorporated in a non-implantable (e.g., wearable) housing and an implantable housing. In FIG. 3, a wearable cardiac stimulation device 100 includes a wearable housing 102 that contains circuitry for implementing the voltage discharge unit 14 and the electrode isolation system 15. The housing 102 may also house the charging source 20, or the charging source may be external to the housing 102. The housing 102 mounts the proximal end 104 of a catheter 106 that can be constructed in the same way as the catheter 12 of FIGS. 1 and 2. At the distal end 108 of the catheter 106 is a tip/ring electrode termination pair 110 comprising a ring electrode 112 and a tip electrode 114 separated by a short insulative stub 116. Although not shown in FIG. 3, electrical leads within the catheter 106 connect the tip/ring electrodes 112/114 to the circuitry in the housing 102.

In FIG. 4, an implantable cardiac stimulation device 200 includes an implantable housing 202 that contains circuitry for implementing the voltage discharge unit 14 and the electrode isolation system 15. The housing 202 preferably also houses the charging source 20, which can be implemented using a battery and a d.c.-d.c. converter to develop the required charging voltage, as described above. The housing 202 mounts the proximal end 204 of a catheter 206 that can be constructed in the same fashion as the catheter 12 of FIG. 1. At the distal end 208 of the catheter 206 is a tip/ring electrode termination pair 210 comprising a ring electrode 212 and a tip electrode 214 separated by a short insulative stub 216.

In either of the embodiments shown in FIGS. 3 and 4, photonic cardio-stimulation functionality can be added by incorporating a photonic pacemaker and/or a photonic cardioverter to the system. More particularly, the housings 102 and 202 of FIGS. 3 and 4 can be provided with photonic pacemaker and/or cardioverter circuitry in addition to the voltage discharge unit 14 and the electrode isolation system 15. The catheter's 106 and 206 could be provided with fiber optic cabling in addition to the electrical leads 26 and 28 so that the catheters function as photonic catheters as well as electrical lead catheters. The fiber optic elements in the catheters 106 and 206 would deliver optical signals that are converted to electrical impulses to drive the electrodes 112/114 and 212/214 for pacing or cardioverter functions. The electrical leads in the catheters 106 and 206 would deliver electrical signals that drive the electrodes 112/114 and 212/214 at higher voltages for defibrillation or other cardio-stimulation purposes. Additional fiber optic elements could be provided in the catheters 106 and 206 to deliver optical sensing signals (such as R-wave amplified signals) from the distal end of each catheter to the respective housings 102 and 204. Note that the sensing signals could be used to control voltage discharge from the voltage discharge unit 14 if the switch 18 is implemented as an automatically controlled device. Reference is hereby made to commonly assigned, copending application Ser. No. 10/014,890, filed Dec. 11, 2001, and entitled “Photonic Pacemaker-Cardiac Monitor.” This application, the contents of which are fully incorporated herein by this reference, is directed to photonic designs for stimulating a heart while simultaneously monitoring one or more biological functions. Such designs could be used in connection with present invention to implement a combined cardiac stimulation device as disclosed herein and a photonic pacemaker and/or cardioverter.

Accordingly, an MRI-safe cardiac stimulation device has been disclosed. As described in detail above, the device can be implemented as a cardiac defibrillator that is designed to operate with an indwelling cardiac catheter powered by a voltage discharge unit. The voltage discharge unit discharges through an electrode isolation system comprising one or more unique spark-gap voltage-activated isolation switches that are adapted to arc in response to the voltage discharge unit output. The invention can thus be used to provide an MRI-safe cardiac defibrillator capable of delivering a pulse of approximately 800 volts (at about 40 joules) for about 10-15 milliseconds via an catheter, and which is particularly suited for use in an MRI theater. MRI compatibility is provided by the electrode isolation system, which disconnects the catheter from the defibrillator circuitry except during defibrillation pulses. The metallic cardiac leads of the catheter are thus protected from the intense MRI electromagnetic fields so they are not able to reach a temperature or deliver voltages capable of damaging the heart or the defibrillator circuitry, as might happen with unprotected cardiac defibrillator leads.

While various embodiments of the invention have been shown and described, it should be apparent that many variations and alternative embodiments could be implemented in accordance with the invention. It is understood, therefore, that the invention is not to be in any way limited except in accordance with the spirit of the appended claims and their equivalents.

I claim:

1. An MRI-safe cardiac stimulation device, comprising:
a voltage discharge unit adapted to provide voltage pulses;
a pair of implantable electrodes connected to deliver voltage pulses from said voltage discharge unit to implanted cardiac tissue; and
an electrode isolation system adapted to electrically isolate said electrodes from said voltage discharge unit during time intervals between said voltage pulses and being responsive to said voltage pulses to connect said voltage discharge unit to said electrodes during said voltage pulses.

2. A device in accordance with claim 1 wherein said electrode isolation system comprises one or more voltage-activated switches adapted to close in response to an applied voltage differential.

3. A device in accordance with claim 2 wherein said one or more voltage-activated switches include a first voltage-activated switch disposed between a first side of said voltage discharge unit and a first one of said electrodes.

4. A device in accordance with claim 3 wherein said one or more voltage-activated switches include a second voltage-activated switch disposed between a second side of said voltage discharge unit and a second one of said electrodes.

5. A device in accordance with claim 2 wherein said one or more voltage-activated switches are adapted to close upon said applied voltage differential being in excess of a voltage induced in said device by an MRI apparatus.

6. A device in accordance with claim 2 wherein said one or more voltage-activated switches are adapted to close upon said voltage differential being less than or equal to a level of said voltage pulses.

7. A device in accordance with claim 2 wherein said one or more voltage-activated switches comprise a gas discharge tube.

8. A device in accordance with claim 1 wherein said electrodes are mounted on a catheter made of a body-compatible material and said electrodes are connected to said voltage discharge unit via electrical leads disposed in said catheter, said leads being made from a material of low magnetic susceptance and sized so as to minimize MRI image disruption.

9. A device in accordance with claim 1 wherein said voltage discharge unit and said electrode isolation system are housed in a housing that is adapted to remain external to a body in which said electrodes are implanted.

10. A device in accordance with claim 1 wherein said voltage discharge unit and said electrode isolation system are housed in an implantable housing.

11. A device in accordance with claim 1 in combination with a photonic pacemaker having an implantable housing carrying said voltage discharge unit and said electrode isolation system, and a photonic catheter carrying said electrodes and electrical leads that deliver said voltage pulses to said electrodes.

12. A device in accordance with claim 1 wherein said voltage discharge unit includes a capacitor adapted for connection to a charging source and a switch adapted to switch between a first switch state in which said charging source is connected to charge said capacitor and a second switch state in which said capacitor is connected to deliver said voltage pulses to said electrodes.

13. A device in accordance with claim 12 wherein said charging source comprises a battery.

14. A device in accordance with claim 12 wherein said switch is adapted for manual control.

15. An MRI-safe cardiac stimulation device, comprising:
pulse generating means for providing voltage pulses;
implantable means for delivering said voltage pulses from said pulse generating means to implanted cardiac tissue; and
electrode isolation means for electrically isolating said implantable means from said pulse generating means during time intervals between said voltage pulses and being responsive to said voltage pulses to connect said pulse generating means to said implantable means during said voltage pulses.

16. A device in accordance with claim 15 wherein said electrode isolation system comprises one or more voltage-activated switches adapted to close in response to an applied voltage differential.

17. A device in accordance with claim 16 wherein said one or more voltage-activated switches include a first voltage-activated switch disposed between a first side of said voltage discharge unit and a first one of said electrodes.

18. A device in accordance with claim 17 wherein said one or more voltage-activated switches include a second voltage-activated switch disposed between a second side of said voltage discharge unit and a second one of said electrodes.

19. A device in accordance with claim 16 wherein said one or more voltage-activated switches are adapted to close upon said applied voltage differential being in excess of a voltage induced in said device by an MRI apparatus.

20. An MRI-safe defibrillator, comprising:
a voltage discharge unit adapted to provide voltage pulses;
a pair of implantable electrodes connected to deliver voltage pulses from said voltage discharge unit to implanted cardiac tissue; and
an electrode isolation system adapted to electrically isolate said electrodes from said voltage discharge unit during time intervals between said voltage pulses and being responsive to said voltage pulses to connect said voltage discharge unit to said electrodes during said voltage pulses.
